(19) United States
(12) Patent Application Publication
(10) Pub. No.: US 2009/0045373 A1
(43) Pub. Date: Feb. 19, 2009

(54) COMPOUNDS, IONIC LIQUIDS, MOLTEN SALTS AND USES THEREOF

(76) Inventors: Amer Hammami, Mont-Royal (CA); Benoit Marsan, Sainte-Julie (CA)

Correspondence Address: BERESKIN AND PARR, 40 KING STREET WEST, BOX 401, TORONTO, ON, M5H 3Y2, CA

(21) Appl. No.: 12/282,198

(22) PCT Filed: Mar. 9, 2007
(86) PCT No.: PCT/CA2007/000390

§ 371 Date: Oct. 23, 2008

Related U.S. Application Data
(60) Provisional application No. 60/780,843, filed on Mar. 10, 2006.

(51) Int. Cl. H01G 9/022; C07D 207/04; C07D 263/08; H01M 8/00; H01M 6/14

(52) U.S. Cl. 252/62.2; 548/579; 548/239; 429/46; 429/336

(57) ABSTRACT

There are provided compounds represented by formula (I):

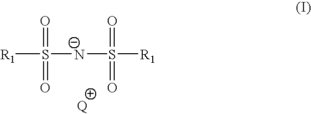

in which R₁ is F, Cl, —N(R₅)₂ or —CN and Q⁺ is selected among various organic cations that include an heterocyle. These compounds are useful as electrolytes, ionic liquids or molten salts.

### FIELD OF THE INVENTION

The present document relates to the field of electrochemistry. In particular, it relates to compounds that are useful as electrolytes such as molten salts or ionic liquids.

### BACKGROUND OF THE INVENTION

An electrolyte in an electrochemical cell may conduct electricity through the movement of ions, charged species, towards an electrode having opposite electrical charge to the ions. Typically, the electrolytes consist of a salt, dissolved in a solvent, which may be water (aqueous solution) or one or more organic compounds (non-aqueous solution). Alternatively, molten salts or ionic liquids, or room temperature molten salts (materials and mixtures which consist of an tonically bound liquid at ambient temperatures) may be used.

In recent years, highly conductive electrolyte salts that are molten at room temperature have been developed for electrochromic windows, variable reflectance mirrors, batteries, capacitors, and other important devices.

U.S. Pat. No. 6,853,472 describes molten salts including lithium or quanternary ammonium cations, and perfluorinated anions selected from the group consisting of trifluoromethylsulfonate (CF₃SO₃⁻), bis(trifluoromethylsulfonyl)imide ((CF₃SO₂)₂N⁻), bis(perfluoroethylsulfonyl)imide ((CF₃CF₂SO₂)₂N⁻) and tris(trifluoromethylsulfonyl)methide ((CF₃SO₂)₃C⁻).

WO 2005/089390 describes methyl-propyl-imidazolium-bis-fluoro-sulfonylimide (MPI-FSI) and ethyl-1-methyl-3-imidazolium-bis-fluoro-sulfonylimide (EMI-FSI) as suitable molten salt electrolytes.

It would therefore be highly desirable to be provided with compounds that would represent an alternative to the compounds previously mentioned.

### SUMMARY OF THE INVENTION

In accordance with one aspect there is provided a compound of formula (I):

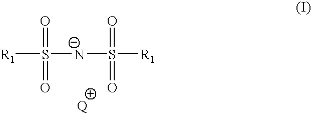

wherein

each of the R₁ is independently F, Cl, —N(R₅)₂, or —CN;

Q⁺ is chosen from

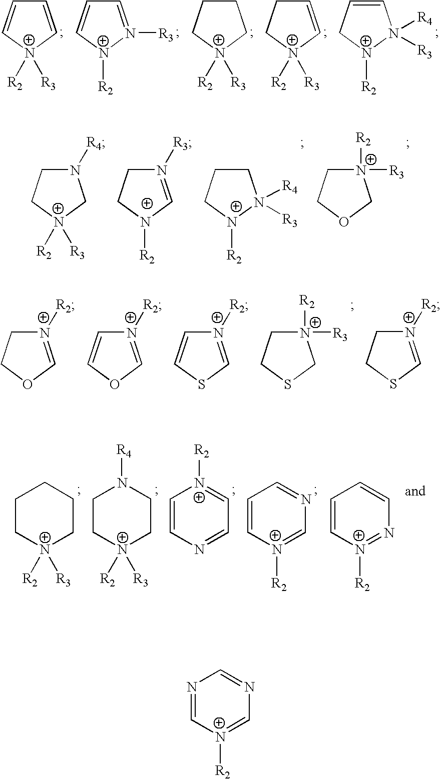

wherein

R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl; and

R₅ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl, an effective protecting group for an amino group,

the heterocycles represented by Q⁺ are as previously presented or substituted with 1 to 3 substituents chosen from of —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂, vinyl, C₄-C₂₀ alkylvinyl, C₄-C₂₀ vinylalkyl, and C₃-C₂₀ expoxyalkyl,
the alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂ₙ₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC₆H₄CₚH₂ₚ₊₁—, and CH₂═CHCₚH₂ₚC₆H₄—,

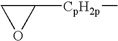

where (1≦n, p≦48),
with the proviso that the compound of formula (I) is different than 1-methyl-1-propylpyrrolidinium imidosulfuryl fluoride.

The compounds previously presented represent a very interesting alternative to the compounds previously proposed in the prior art. In fact, these compounds can be simply and rapidly prepared at low costs.

In accordance with another aspect there is provided a process for preparing a compound of formula (I):

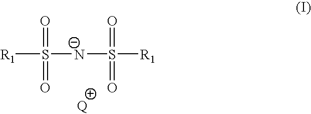

wherein

each of the R₁ is independently F, Cl, —N(R₅)₂, or —CN,

Q⁺ is chosen from

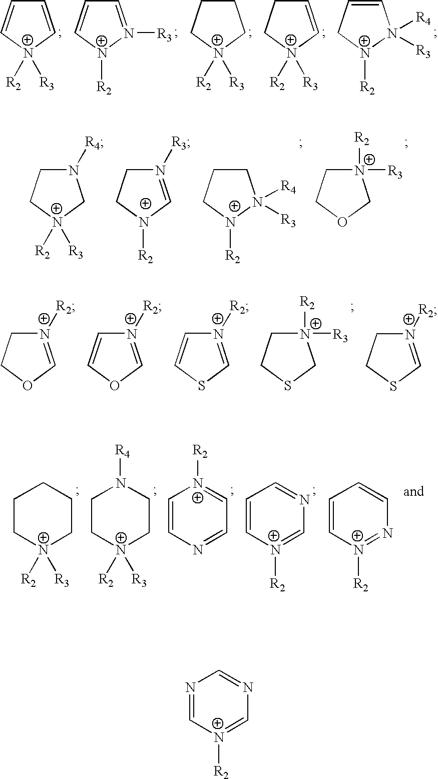

wherein

R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl; and

R₅ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl, an effective protecting group for an amino group,

the heterocycles represented by Q⁺ are as previously presented or substituted with 1 to 3 substituents chosen from —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂, vinyl, C₄-C₂₀ alkylvinyl, C₄-C₂₀ vinylalkyl, and C₃-C₂₀ expoxyalkyl,
the alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂ₙ₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC₆H₄CₚH₂ₚ₊₁—, and CH₂═CHCₚH₂ₚC₆H₄

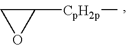

where (1≦n, p≦48)
comprising the step of reacting together a compound of formula (V) and a compound of formula (VI):

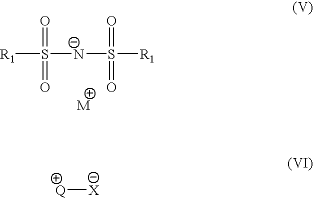

wherein

R₁ and Q are as previously defined;

M⁺ is chosen from Li⁺, Na⁺, K⁺, and Cs⁺

X⁻ is chosen from F⁻, Cl⁻, Br⁻, I⁻, CH₃COO⁻, PhCH₂COO⁻, CN⁻, CF₃COO⁻, SO₄²⁻, CF₃SO₃⁻, BF₄⁻, PF₆⁻, NO₃⁻, ClO₄⁻, SbF₆⁻, and RuO₄⁻.

Such a process is useful and efficient to prepare, at low costs, compounds of general formula (I). This process is simple and can easily be carried out.

According to another aspect, there is provided a process for preparing a compound of formula (Ia):

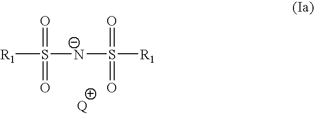

wherein

each of the R₁ is independently F or Cl,

Q⁺ is chosen from

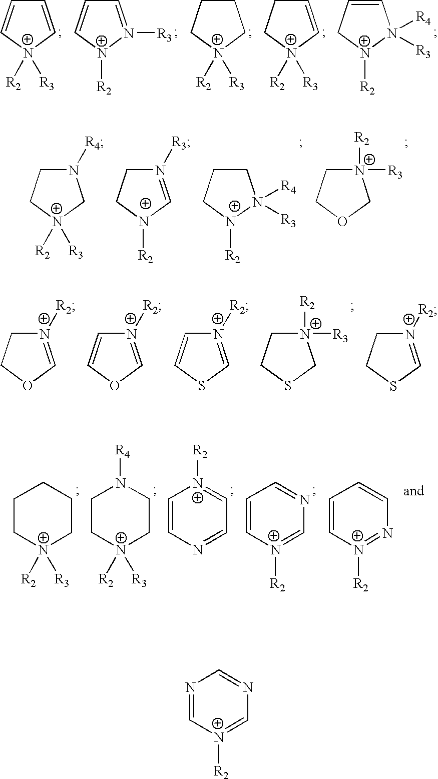

wherein

R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl; and

R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl, the heterocycles represented by Q⁺ are as previously presented or substituted with 1 to 3 substituents chosen from —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂, vinyl, C₄-C₂₀ alkylvinyl, C₄-C₂₀ vinylalkyl, and C₃-C₂₀ expoxyalkyl,

the alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂ₙ₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC6H₄CₚH₂ₚ₊₁—, and CH₂═CHCₚH₂ₚC₆H₄—,

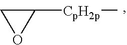

where (1≦n, p≦48)

comprising the step of reacting a compound of formula (II):

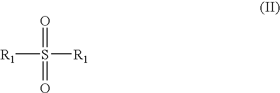

wherein each of the R₁ is as previously defined,

with a compound of formula (III):

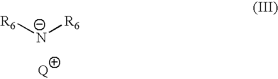

wherein

Q⁺ is as previously defined for formula (Ia); and

each of R₆ is independently H, Li, Na, K, Cs, or (R₇)₃Si—, each of the R₇ being independently a C₁-C₁₂ alkyl.

According to another aspect, there is provided a process for preparing a compound of formula (Ib):

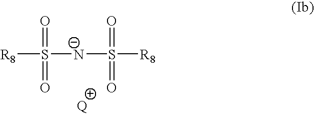

wherein

R₈ is F; and

Q⁺ is chosen from

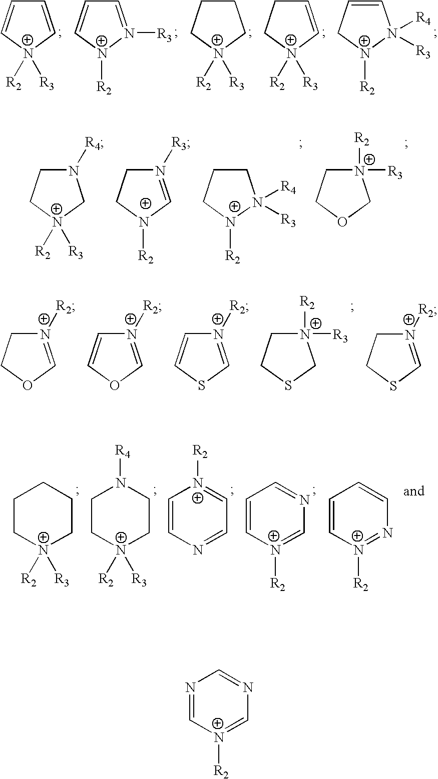

wherein

R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl; and

R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl, the heterocycles represented by Q⁺ are as previously presented or substituted with 1 to 3 substituents chosen from —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂ vinyl, C₄-C₂₀ alkylvinyl, C₄-C₂₀ vinylalkyl, and C₃-C₂₀ expoxyalkyl,

the alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂ₙ₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC₆H₄CₚH₂ₚ₊₁—, and CH₂═CHCₚH₂ₚC₆H₄—

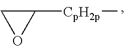

where (1≦n, p≦48)
comprising the steps of:
a) reacting SO₂Cl₂ with a compound of formula (III):

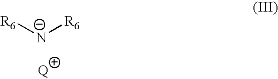

wherein

Q⁺ is as previously defined for formula (Ib); and

each of the R₆ is independently H, Li, Na, K, Cs, or (R₇)₃Si—,

each of the R₇ being independently a C₁-C₁₂ alkyl
so as to obtain a compound of formula (Ic);

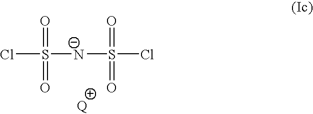

wherein

Q⁺ is as previously defined for formula (Ib); and

b) reacting the compound of formula (Ic) with a compound of formula MF, wherein M is Li, Na, K, or Cs, so as to obtain the compound of formula (Ib).

According to another aspect, there is provided a process for preparing a compound of formula (Ib):

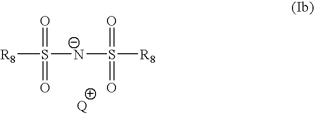

wherein

R₈ is F; and

Q⁺ is chosen from

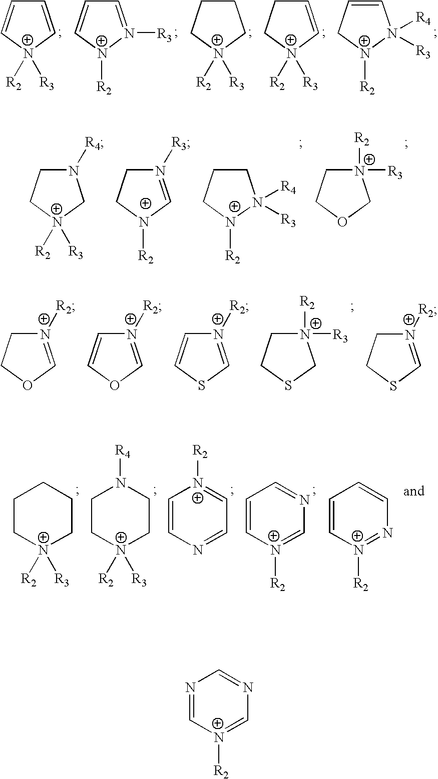

wherein

R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl; and

R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl, the heterocycles represented by Q⁺ are as previously presented or substituted with 1 to 3 substituents chosen from —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂, vinyl, C₄-C₂₀ alkylvinyl, C₄-C₂₀ vinylalkyl, and C₃-C₂₀ expoxyalkyl,

the alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂ₙ₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC₆H₄CₚH₂ₚ₊₁—, and CH₂═CHCₚH₂ₚC₆H₄—

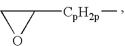

where (1≦n, p≦48),
comprising the steps of:
a) reacting a compound of formula (IIa):

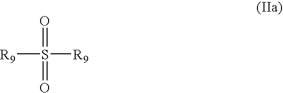

wherein

each of the R₉ is independently Cl, Br, or I
with a compound of formula (IIIa):

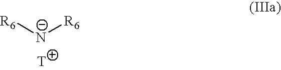

wherein

T⁺ is Li⁺, Na⁺, K⁺, Cs⁺ or H⁺ and

each of the R₆ is independently H, Li, Na, K, Cs, or (R₇)₃Si—, each of the R₇ being independently a C₁-C₁₂ alkyl.

so as to obtain a compound of formula (VII);

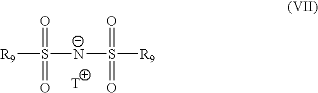

wherein

each of the R₉ is as previously defined for formula (Ia); and

T⁺ is as previously defined for formula (IIIa); and
b) reacting the compound of formula (VII) with a compound of formula Q-R₈, wherein Q and R₈ are as previously defined in formula (Ib), so as to obtain the compound of formula (Ib).

The term “alkyl” as used herein refers to linear or branched radicals. Examples of such radicals include, but are not limited to, methyl, ethyl, n-propyl, isopropyl, n-butyl, isobutyl, sec-butyl, tert-butyl, pentyl, iso-amyl, hexyl and the like. For example, the alkyl can be a methyl.

The term “aryl” has used herein refers to a cyclic or polycyclic aromatic ring. The aryl group can be a phenyl or napthyl.

The term “heteroaryl” has used herein refers to an aromatic cyclic or fused polycyclic ring system having at least one heteroatom chosen from N, O, and S. For example, the heteroaryl groups include, but are not limited to, furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, among others.

The term “heterocyclyl” includes non-aromatic rings or ring systems that contain at least one ring having at least one hetero atom (such as nitrogen, oxygen or sulfur). For example, this term can include all of the fully saturated and partially unsaturated derivatives of the above mentioned heteroaryl groups. Examples of heterocyclic groups include, without limitation, pyrrolidinyl, tetrahydrofuranyl, morpholinyl, thiomorpholinyl, piperidinyl, piperazinyl, thiazolidinyl, isothiazolidinyl, and imidazolidinyl.

In the compounds and processes previously presented, Q⁺ can be chosen from

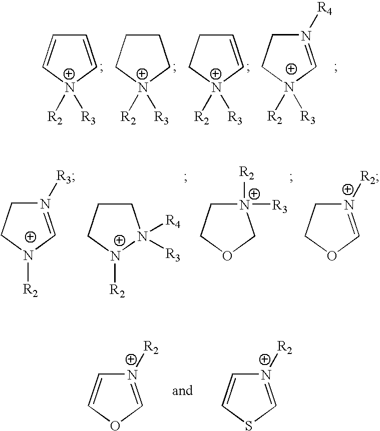

Alternatively, Q⁺ can be chosen from:

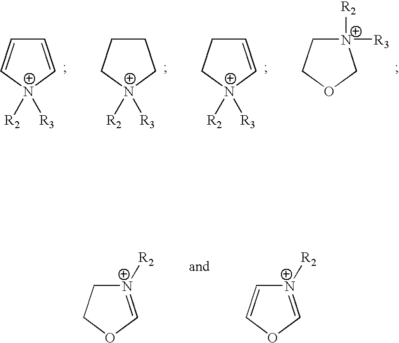

According to another example Q⁺ can be chosen from

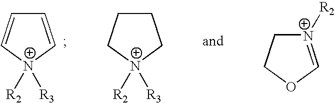

R₂ can be a C₁-C₂₀ alkyl which is linear or branched or a C₃-C₁₂ cycloalkyl. According to one example, R₂ can be a C₁-C₂₀ alkyl which is linear or branched. According to another example, R₂ can be a C₁-C₈ alkyl which is linear. R₃ can be a C₁-C₂₀ alkyl which is linear or branched or a C₃-C₁₂ cycloalkyl. According to one example, R₃ can be a C₁-C₂₀ alkyl which is linear or branched. According to another example, R₃ is a C₁-C₈ alkyl which is linear. R₄ can be a C₁-C₂₀ alkyl which is linear or branched or a C₃-C₁₂ cycloalkyl. According to one example, R₄ can be a C₁-C₂₀ alkyl which is linear or branched. According to another example, R₄ can be a C₁-C₈ alkyl which is linear. According to a further example, R₄ can be a C₁-C₄ alkyl which is linear.

The compounds previously presented can have a conductivity of at least 0.0001 mS cm⁻¹. For example, the conductivity can be of at least 1 mS cm⁻¹, or of at least 10 mS cm⁻¹. Alternatively, they can have a conductivity of about 0.0001 to about 100 mS cm⁻¹. The compounds can have a melting point below 100° C. For example, the melting point can be below 40° C., or below 25° C. Alternatively, the compounds can have a melting point of about 0° C. to about 100° C. For example, the R₁ group can be a halogen atom. According to one example, R₁ is F or Cl. According to another example, R₁ is F.

The compounds previously presented can be used as a molten salt, an ionic liquid or an electrolyte. These compounds can also be used in an electrochemical device such as a battery.

In the process for preparing the compounds represented by formula (I), the reaction can be carried out in water so that the so-obtained product of formula (I) precipitates and the so-formed byproduct of formula M⁺X) is at least substantially soluble. For example, M⁺ can be K⁺. For example, X⁻ can be F⁻, Cl⁻, Br⁻, or I⁻. According to another example, X⁻ is Cl⁻, or Br⁻. Each of the R₁ can be a halogen atom. According to another example, R₁ can be Cl⁻ or F⁻. According to another example, R₁ can be F⁻.

In the process for preparing compounds represented by formula (Ia), the compound of formula (III) can be a compound of formula (IV):

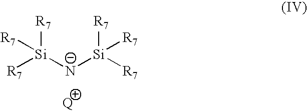

wherein

Q⁺ is as previously defined in formula (I); and

each of the R₇ is independently a C₁-C₁₂ alkyl.

For example, each of the R₇ can be the same. According to one example, R₇ can be methyl. The compounds of formulas (II) and (III) can be reacted together at a temperature of about −78 to about 110° C. The temperature can be for example about −5 to about 25° C., or about 15 to about 25° C. R₁ can be F or Cl. According to one example, R₁ can be F. In the process for preparing compounds represented by formula (Ib), step (a) can be carried out at a temperature of about −78 to about 110° C. For example, the temperature can be about −5 to about 25° C. or about 15 to about 25° C. Step (b) can be carried out in the presence of an aprotic solvent. For example, the aprotic solvent can be a polar solvent such as nitromethane or acetonitrile. According to one example, the compound of formula (III) can be a compound of formula (IV):

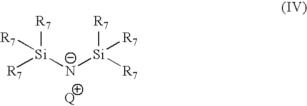

wherein

Q⁺ is as previously defined in formula (Ib); and

each of the R₇ is independently a C₁-C₁₂ alkyl.

Each of the R₇ can be the same. For example, R₇ can be a methyl.

In the process for preparing compounds represented by formula (Ib), the compound of formula (IIIa) can be a compound of formula (IVa):

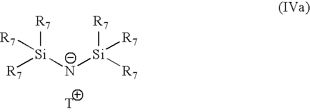

wherein

T⁺ is as previously defined in formula (IIIa); and

each of the R₇ is independently a C₁-C₁₂ alkyl.

Each of the R₇ can be the same. For example, each of the R₇ can be a methyl.

In accordance with another aspect there is provided a molten salt comprising a compound as defined in the present invention.

In accordance with another aspect there is provided an ionic liquid comprising a compound as defined in the present invention.

In accordance with another aspect, there is provided an electrolyte comprising a compound as defined in the present invention.

In accordance with another aspect, there is provided an electrochemical device comprising a compound as defined in the present invention.

In accordance with another aspect, there is provided a battery comprising a compound as defined in the present invention.

In accordance with another aspect, there is provided a method of using a compound as previously defined, which comprises contacting the compound with electrodes and using it as an electrolyte.

In accordance with another aspect, there is provided a method of using a compound as previously defined, which comprises introducing the compound in the manufacture of a proton exchange membrane.

The compounds previously described can be used in many applications. For example, they can be used as solvents for organic and organometallic syntheses and catalysis. They can also be used as electrolytes (for example in electrochemistry or in fuel and solar cells), as lubricants, as a stationary phase for chromatography, as matrices for mass spectrometry, supports for the immobilization of enzymes, in separation technologies, as liquid crystals, templates for the synthesis of mesoporous, nano-materials and ordered films, materials for embalming and tissue preservation, etc.

The compounds previously mentioned can be used in various solutions (dry cleaning, metal extraction, personal care, embalming, household products, coatings, etc.) and in electrochemistry (batteries, solar panel, ion propulsion, fuel cells, electro-optics, etc.). The can also be used in view of their various interesting properties for heat transfer or as lubricants. They can also be used in drug delivery, biomass processing, biocides etc.

The compounds previously mentioned can also be useful for preparing compositions for lithium-ions batteries.

In accordance with another aspect there is provided a composition comprising a compound of formula (I) and a compound of formula (VIII):

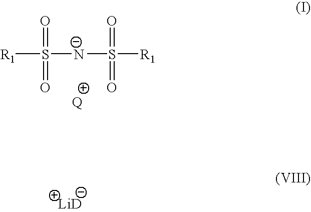

wherein

each of the R₁ is independently F, Cl, —N(R₅)₂, or —CN;

Q⁺ is chosen from

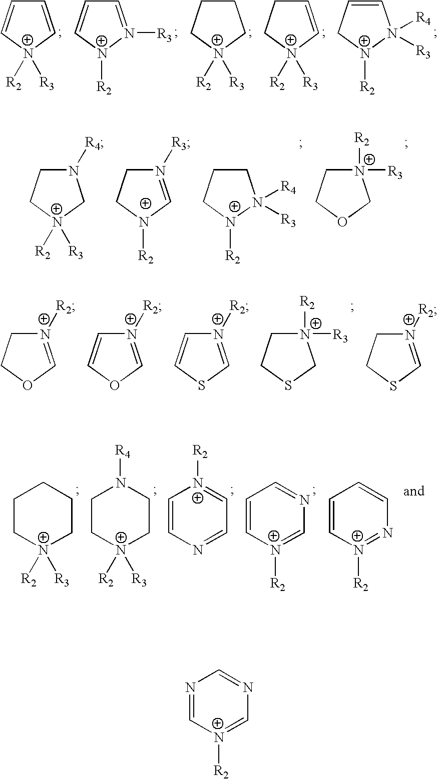

wherein

D is chosen from CF₃SO₃—, (FSO₂)₂N—, (CF₃SO₂)₂N—, (CF₃CF₂SO₂)₂N—, (CF₃SO₂)₃C—, PF₆⁻, CF₃COO⁻, AsF₆⁻, CH₃COO⁻, (CN)₂N⁻, NO₃⁻, BF₄⁻, ClO₄⁻, (C₈H₁₆SO₂)₂N⁻, and C₃H₃N₂⁻

R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl;

R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl; and

R₅ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl, an effective protecting group for an amino group,

the heterocycles represented by Q⁺ are as previously presented or substituted with 1 to 3 substituents chosen from of —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂, vinyl, C₄-C₂₀ alkylvinyl, C₄-C₂₀ vinylalkyl, and C₃-C₂₀ expoxyalkyl,
the alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC₆H₄CₚH₂ₚ₊₁—, and CH₂═CHCₚH₂ₚC₆H₄—,

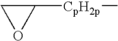

where (1≦n, p≦48)

In accordance with another aspect, there is provided a method of using a compound as previously defined, which comprises mixing the compound with a compound of formula (VIII) so as to obtain a mixture and using said mixture as an electrolyte, for example in a lithium-ion battery.

### DETAILED DESCRIPTION OF THE INVENTION

The following examples are given in a non-limitative manner.

### EXAMPLE 1

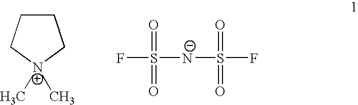

### Compound 1

2 g (14.81 mM) of sulfuryl chloride are charged under argon into a 500 mL flask and mixed with 50 mL of anhydrous acetonitrile. Then, the mixture is cooled at −20° C. 14.81 mL of a potassium hexamethyldisilazane (KHMDS) solution (0.5 M in toulene) is added dropwise over 5 minutes at −20° C. under argon. The mixture is stirred at room temperature for 12 h. Then, the solvent is removed under vacuum and the resulting brown crude is dissolved in 100 mL acetonitrile and mixed with 1.72 g (29.08 mM) of anhydrous KF. The reaction mixture is heated and stirred over 12 h. Then, the solid particles are filtered-out and the solvent is removed under vacuum and replaced by 100 mL of distilled water. The aqueous solution is charged into a 500 mL flask and mixed with 100 mL of an aqueous solution of 1.68 g (7.4 mM) of N,N-dimethyl-pyrrolidinium iodide. The resulting compound 1 is then extracted by dichloromethane and isolated in pure form.

### EXAMPLE 2

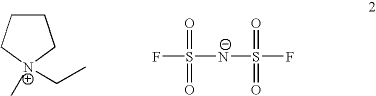

### Compound 2

Potassium bis(fluoromethanesulfonimide) KFSI is prepared as previously described and 2.2 g (10 mM) of this compound are used to prepare an aqueous solution by charging it into a 500 mL flask and dissolving it into 50 mL of distilled water. 2.41 g (10 mM) of N,N-ethylmethylpyrrolidinium iodide is dissolved into 50 mL of distilled water and then mixed with KFSI solution. The N,N-ethylmethylpyrrolidinium iodide exchanges anions with KFSI in water. The Potassium iodide stays in the aqueous phase and the desired molten salt 2 is decanted. The organic layer is decanted, extracted with 40 mL of CH₂Cl₂ and then washed with 80 mL of distilled H₂O and dried over anhydrous MgSO₄. After concentration with a rotative evaporator, the translucent ionic liquid obtained is dried under vacuum at 60° C. for 3 hours. Its purity is confirmed by NMR (¹H, ¹³C, ¹⁹F) and cyclic voltammetry.

### EXAMPLE 3

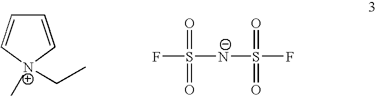

### Compound 3

Potassium bis(fluoromethanesulfonimide) KFSI is prepared as previously described and 2.2 g (10 mM) of this compound are used to prepare an aqueous solution by charging it into a 500 mL flask and dissolving it into 50 mL of distilled water. 2.37 g (10 mM) of N,N-ethylmethylpyrrolium iodide was dissolved into 50 mL of distilled water and then mixed with KFSI solution. The N,N-ethylmethylpyrrolium iodide exchanges anions with KFSI in water. The potassium iodide stays in the aqueous phase and the desired molten salt 3 is decanted. The organic layer was decanted, extracted with 40 mL of CH₂Cl₂ and then washed with 80 mL of distilled H₂O and dried over anhydrous MgSO₄. After concentration with a rotative evaporator, the translucent ionic liquid obtained is dried under vacuum at 60° C. for 3 hours. Its purity is confirmed by NMR (1H, 13C, 19F) and cyclic voltammetry.

### EXAMPLE 4

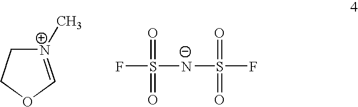

### Compound 4

Potassium bis(fluoromethanesulfonimide) KFSI is prepared as previously described and 2.2 g (10 mM) of this compound are used to prepare an aqueous solution by charging it into a 500 mL flask and dissolving it into 50 mL of distilled water. 2.13 g (10 mM) of, N-methyloxazolinium iodide is dissolved into 50 mL of distilled water and then mixed with KFSI solution. The N-methyloxazolinium iodide exchanges anions with KFSI in water. The potassium iodide stays in the aqueous phase and the desired molten salt 4 is decanted. The organic layer is decanted, extracted with 60 mL of CH₂Cl₂ and then washed with 100 mL of distilled H₂O and dried over anhydrous MgSO₄. After concentration with a rotative evaporator, the translucent ionic liquid obtained is dried under vacuum at 60° C. for 3 hours. Its purity is confirmed by NMR (¹H, ¹³C, ¹⁹F) and cyclic voltammetry.

The person skilled in the art would clearly recognize that all the references cited in this application are hereby incorporated by references. The person skilled in the art would also recognize that various modifications, adaptations, and variations may be brought to the previously presented preferred embodiments without departing from the scope of the following claims.

1. A compound of formula (I): 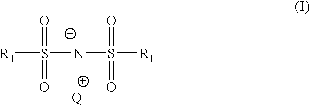
wherein
each of the R₁ is independently F, Cl, —N(R₅)₂, or —CN,
Q⁺ is selected from the group consisting of 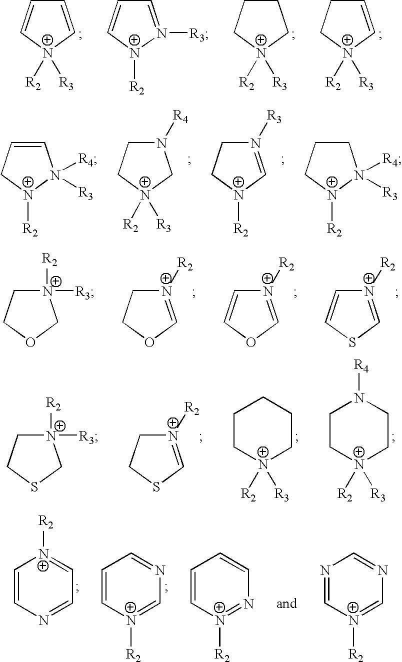
wherein
R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl; and
R₅ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or an effective protecting group for an amino group,
said heterocycles represented by Q⁺ are as previously presented or substituted with 1 to 3 substituents chosen from —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂, vinyl, C₄-C₂₀ alkylvinyl, C₄-C₂₀ vinylalkyl, or C₃-C₂₀ epoxyalkyl,
said alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂ₙ₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC₆H₄CₚH₂ₚ₊₁—, and CH₂═CHCₚH₂ₚC₆H₄—, 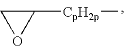
where
with the proviso that when at least one of said R₁ is F, Q⁺ is different than 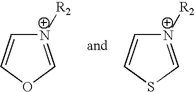
and that said compound of formula (I) is different than 1-methyl-1-propylpyrrolidinium imidosulfuryl fluoride.

2. The compound of claim 1, wherein each of said R₁ is F or Cl.

3. The compound of claim 1, wherein each of said R₁ is F.

4. The compound of claim 3, wherein Q⁺ is chosen from

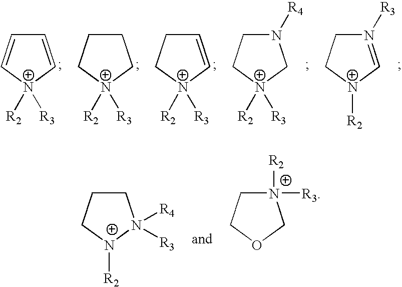

5. The compound of claim 3, wherein Q⁺ is chosen from

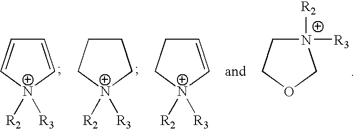

6. (canceled)

7. The compound of claim 3, wherein Q⁺ is chosen from

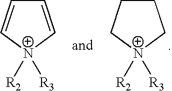

8. The compound of claim 3, wherein Q⁺ is

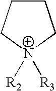

9. The compound of claim 5, wherein R₂ is a C₁-C₂₀ alkyl which is linear or branched or a C₃-C₁₂ cycloalkyl, and R₃ is a C₁-C₂₀ alkyl which is linear or branched or a C₃-C₁₂ cycloalkyl.

10. The compound of claim 5, wherein R₂ is a C₁-C₂₀ alkyl which is linear or branched, and R₃ is a C₁-C₂₀ alkyl which is linear or branched.

11. The compound of claim 8, wherein R₂ is a C₁-C₈ alkyl which is linear, and R₃ is a C₁-C₈ alkyl which is linear.

12. (canceled)

13. (canceled)

14. The compound of claim 8, wherein R₂ and R₃ are identical, and they represent a C₁-C₈ alkyl which is linear.

15. The compound of claim 14, wherein R₂ ═R₃=—CH₃.

16. The compound of claim 11, wherein R₂=—CH₃, and R₃=—CH₂CH₃.

17. (canceled)

18. (canceled)

19. (canceled)

20. The compound of claim 1, wherein said compound has a conductivity of at least 1 mS cm⁻¹.

21. The compound of claim 1, wherein said compound has a conductivity of at least 10 mS cm⁻¹.

22. (canceled)

23. (canceled)

24. The compound of claim 20, wherein said compound has a melting point below 40° C.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method of using a compound as defined in claim 1, comprising mixing said compound with a compound of formula (VIII) 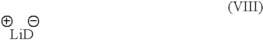
wherein
D is chosen from CF₃SO₃—, (FSO₂)₂N—, (CF₃SO₂)₂N—, (CF₃CF₂SO₂)₂N—, (CF₃SO₂)₃C—, PF₆⁻, CF₃COO⁻, AsF₆⁻, CH₃COO⁻, (CN)₂N⁻, NO₃⁻, BF₄⁻, ClO₄⁻, (C₈H₁₆SO₂)₂N⁻, and C₃H₃N₂⁻,
so as to obtain a mixture and using said mixture as an electrolyte.

41. (canceled)

42. A process for preparing a compound of formula (Ia): 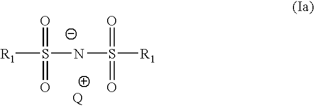
wherein
each of said R₁ is independently F or Cl,
Q⁺ is chosen from 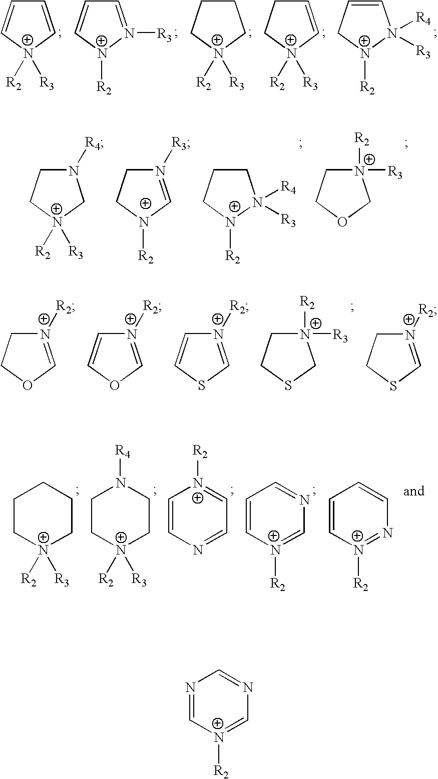
wherein
R₂ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R₃ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl; and
R₄ is a hydrogen atom, a C₁-C₂₀ alkyl which is linear or branched, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl,
said heterocycles represented by Q⁺ are as previously presented or substituted with 1 to 3 substituents chosen from —NO₂, —CN —OH, —CF₃—COR₄, —SH, —OMe, —OCH₂Ph, —SMe, —SPh, —SCH₂Ph, —COOH, —COOR₄, —NH₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₂-C₁₂ heteroaryl, C₁-C₁₂, vinyl, C₄-C₂₀ alkylvinyl, C₄-C₂₀ vinylalkyl, and C₃-C₂₀ epoxyalkyl,
said alkyl, cycloalkyl, heterocyclyl, alkenyl, alkynyl, aryl, aralkyl, alkylaryl, and heteroaryl being unsubstituted or substituted with 1 to 3 substituents chosen from F, Cl, Br, I, OH, a C₁-C₆ alkoxy, a C₁-C₆ hydroxy alkyl, NO₂, CN, CF₃, SO₃⁻, CₙF₂ₙ₊₁, C₁-C₁₂ alkyl which is linear or branched, C₆-C₁₂ aryl, CₙH₂ₙ₊₁, Ph₂P(O)—, Ph₂P—, Me₂P(O)—, Me₂P, Ph₂P(S), Me₂P(S), Ph₃P═N—, Me₃P═N—, C₆H₅CₚH₂ₚ—, CₚH₂ₚ₊₁C₆H₄—, CₚH₂ₚ₊₁C₆H₄CₙH₂ₙ—, CH₂═CHCₚH₂ₚ—, CH₂═CHC₆H₅—, CH₂═CHC₆H₄CₚH₂ₚ₊₁—, and CH₂═CHCₚH₂ₚC₆H₄— 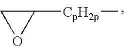
where,
comprising the step of reacting a compound of formula (II): 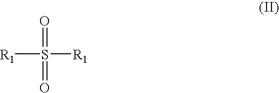
wherein each of said R₁ is as previously defined,
with a compound of formula (III): 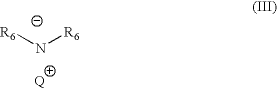
wherein
Q⁺ is as previously defined for formula (Ia); and
each of said R₆ is independently H, Li, Na, K, Cs, or (R₇)₃Si—, each of said R₇ being independently a C₁-C₁₂ alkyl.

43. The process of claim 42, wherein said compound of formula (III) is a compound of formula (IV): 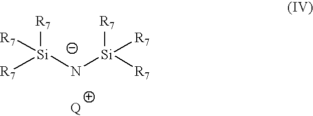
wherein
Q⁺ is as previously defined in formula (I); and
each of said R₇ is independently a C₁-C₁₂ alkyl.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. A composition comprising a compound as defined in claim 1 and a compound of formula (VIII): 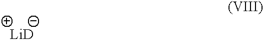
wherein
D is chosen from CF₃SO₃—, (FSO₂)₂N—, (CF₃SO₂)₂N—, (CF₃CF₂SO₂)₂N—, (CF₃SO₂)₃C—, PF₆⁻, CF₃COO⁻, AsF₆⁻, CH₃COO⁻, (CN)₂N⁻, NO₃⁻, BF₄⁻, ClO₄⁻, (C₈H₁₆SO₂)₂N—, and C₃H₃N₂⁻.

94. (canceled)
